(19) United States
(12) Patent Application Publication
(10) Pub. No.: US 2013/0266508 A1
(43) Pub. Date: Oct. 10, 2013

(54) THERMOSENSITIVE HYDROGEL FOR COATING RADIOISOTOPE AND CHEMOTHERAPEUTIC AGENT TO TREAT CANCER AND METHOD FOR PREPARING THE SAME

(71) Applicants: Tsai-Yueh Luo, TAOYUAN COUNTY (TW); Cheng-Liang Peng, TAOYUAN COUNTY (TW); Ming-Jium Shieh, TAOYUAN COUNTY (TW); Ying-Hsia Shih, TAOYUAN COUNTY (TW); Chung-Hsin Yeh, TAOYUAN COUNTY (TW); I-Chung Tang, TAOYUAN COUNTY (TW)

(72) Inventors: Tsai-Yueh Luo, TAOYUAN COUNTY (TW); Cheng-Liang Peng, TAOYUAN COUNTY (TW); Ming-Jium Shieh, TAOYUAN COUNTY (TW); Ying-Hsia Shih, TAOYUAN COUNTY (TW); Chung-Hsin Yeh, TAOYUAN COUNTY (TW); I-Chung Tang, TAOYUAN COUNTY (TW)

(73) Assignee: ATOMIC ENERGY COUNCIL-INSTITUTE OF NUCLEAR ENERGY RESEARCH, TAOYUAN COUNTY (TW)

(21) Appl. No.: 13/441,961

(22) Filed: Apr. 9, 2012

(51) Int. Cl. A61K 51/12; A61K 31/704; A61P 35/00; A61K 31/4745; A61K 51/00; A61K 47/34; A61K 9/00; A61K 31/337

(52) U.S. Cl. 424/1.11; 424/400; 514/34; 514/449; 514/283; 514/772.7

(57) ABSTRACT

A thermosensitive hydrogel for coating radioisotopes and chemotherapeutic agents to treat cancer and a method for preparing the same are revealed. The anticancer drugs such as radiopharmaceuticals or chemotherapeutic agents are coated with the hydrogel formed by PCL-PEG-PCL. By the feature of the hydrogel body that changes from liquid phase at low storage temperature to gel form at body temperature, not only the anticancer drugs can be injected into the human body and reaching the treatment site smoothly but the treatment time of brachytherapy is also extended. Thus the side effects of cancer therapy are significantly reduced.

### BACKGROUND OF THE INVENTION

1. Field of the Invention

The present invention relates to a thermosensitive hydrogel for treatment of cancers and a method for preparing the same, especially to a thermosensitive hydrogel for coating radioisotopes and chemotherapeutic agents, and treatment of cancer and a method for preparing the same.

2. Descriptions of Related Art

The main cancer treatment options available now include chemotherapy, radiation therapy, and surgery.

The surgery removes the tumor and most of affected areas of the body. But for cancers such as blood caner or metastases that are not confined in a specific area, surgery doesn't play a major role in their management.

Chemotherapy is using anticancer drugs to kill cancer cells. The treatment plan depends on the particular type of cancer and the condition of the patient. The chemotherapy can be performed before surgery or follow up treatment after surgery, applied in combination with radiation therapy or by it's self. However, chemotherapeutic agents have cytotoxicity and cause unpleasant side effect.

Radiotherapy or radiation therapy is the treatment of cancer and other diseases with ionizing radiation including high energy waves or particles. Radiation kills or stunts the growth of cancerous cells. The cancer cells grow and divide much faster than normal cells. Thus radiation therapy can control many cancers in sufficient does. However, the high-dose radiation rays that injury cancer cells also cause damage to normal cells during the course of treatment so that serious side effects occur.

Thus the most important issue for cancer therapy at this stage is to develop a more effective way to diagnose and treat cancers. Brachytherapy is an advanced cancer treatment. Radioisotopes are placed in or around the tumor site, giving a high radiation dose to the tumor site while reducing damage to the surrounding normal tissues. Some tumors are generally treated with brachytherapy including buccal cancer, neck cancer, uterus cancer, vaginal cancer, cervical cancer, and prostate cancer. As to internal radiation therapy for liver cancer patients, drugs are applied by Transcatheter Arterial Embolization (TAE). However, TAE requires advanced surgical techniques and high-cost medical equipment.

Thus the present invention reveals a drug delivery module for brachytherapy. When the tumor site is treated by high-dose radiation or chemotherapeutic agents, the damage to the surrounding normal tissues can be reduced. Moreover, the instrument or equipment needed is simple and easy to get. This is beneficial to the applications of Brachytherapy.

### SUMMARY OF THE INVENTION

Therefore it is a primary object of the present invention to provide a thermosensitive hydrogel for coating radioisotopes and chemotherapeutic agents to treat cancer and a method for preparing the same. The radiopharmaceuticals and nanosized chemotherapeutic agents are coated with polymer hydrogel. Due to temperature difference between storage temperature and body's temperature, the hydrogel is in a liquid form before being injected into human bodies while the hydrogel changes into gel phase after being injected into human bodies. Thus the drugs can stay in organs or the treatment site being injected for a longer time, without diffusing into other normal tissues.

It is another object of the present invention to provide a thermosensitive hydrogel for coating radioisotopes and chemotherapeutic agents to treat cancer and a method for preparing the same that concentrate radiation dose of the radiation therapy and chemical agents used in chemotherapy on the tumor site to reduce their negative effects on the surrounding normal tissues.

It is a further object of the present invention to provide a thermosensitive hydrogel for coating radioisotopes and chemotherapeutic agents to treat cancer and a method for preparing the same. The thermosensitive hydrogel will be degraded and metabolized gradually in the human body, without any side effect.

It is a further object of the present invention to provide a thermosensitive hydrogel for coating radioisotopes and chemotherapeutic agents to treat cancer and a method for preparing the same. The hydrogel with good bio-compatibility can reduce side effects on patients, and also reduce the risk of treatment complications.

In order to achieve the above objects, a thermosensitive hydrogel for treatment of cancers of the present invention includes a hydrogel body and a plurality of anticancer drug molecules encapsulated in the hydrogel body. The hydrogel body is a polymer compound named Polycaprolactone-Polyethylene glycol-Polycaprolactone (PCL-PEG-PCL). A method for preparing a thermosensitive hydrogel includes following steps. Synthesize a polymer compound, PCL-PEG-PCL. Then prepare a drug molecule. Mix the polymer compound with the drug molecule and the temperature is increased and decreased repetitively during the mixing processes.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is a schematic drawing showing composites of an embodiment according to the present invention;

FIG. 2 is a figure showing comparisons of features of an embodiment according to the present invention;

FIG. 3 is a flow chart showing steps of preparation of an embodiment according to the present invention;

FIG. 4A shows chemical formulas for a chemical reaction of an embodiment according to the present invention;

FIG. 4B shows chemical formulas for a chemical reaction of an embodiment according to the present invention;

FIG. 5 is a flow chart showing steps of preparation of an embodiment according to the present invention;

FIG. 6 shows results of an effect test of an embodiment according to the present invention.

### DETAILED DESCRIPTION OF THE PREFERRED EMBODIMENT

As to conventional cancer treatment methods, the therapeutic effect is reduced due to strong side effect or failure of concentrating drugs or focusing radiation on the tumor site. The present invention overcomes these shortcomings by thermosensitive hydrogel used as coating material for anticancer drugs to improve the effects of brachytherapy.

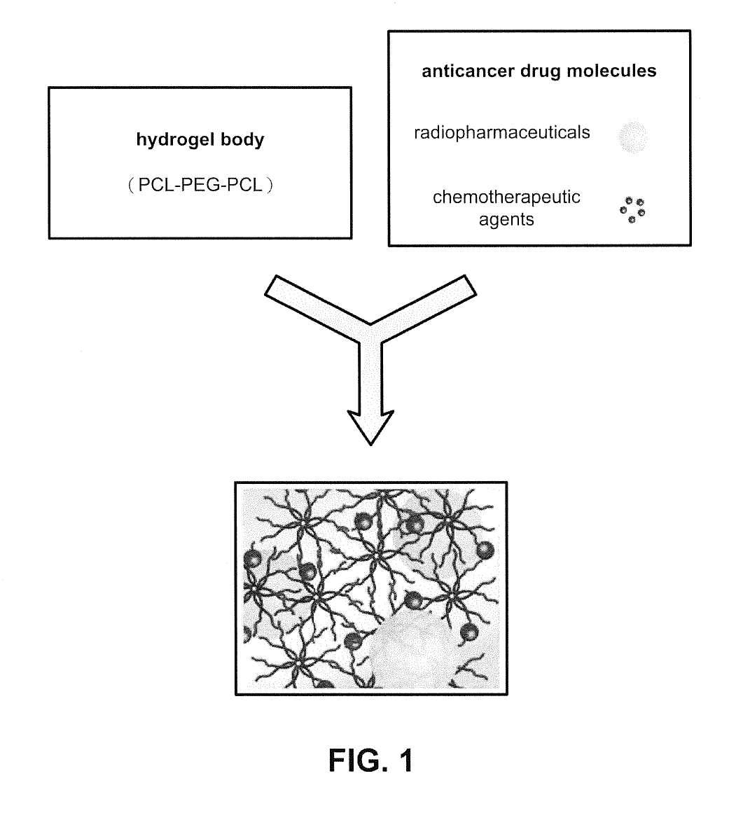

Refer to FIG. 1, a thermosensitive hydrogel for treatment of cancers includes a hydrogel body that is a polymer compound named Polycaprolactone-Polyethylene glycol-Polycaprolactone (PCL-PEG-PCL). The number-average molecular weight of the polymer compound is about 2500. The ratio of the number-average molecular weight between PCL and PEG is 3:2.

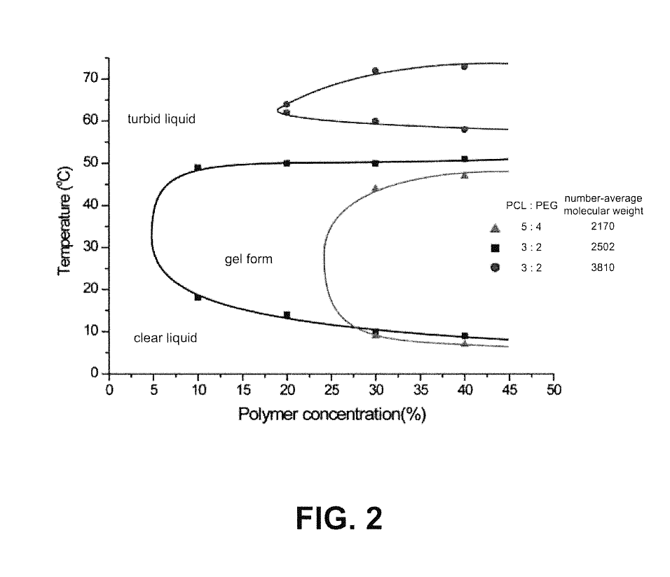

These are characteristics of the polymer compound. According to such ratio, PCL-PEG-PCL in lower concentration is in gel form at normal body temperature of humans—37 degrees Celsius. Refer to FIG. 2, the physical properties of the polymer match the above condition when the number-average molecular weight of PCL-PEG-PCL is about 2500 and the ratio of the number-average molecular weight of PCL to that of PEG is 3:2. Moreover, the polymer compound feature on that it is a clear liquid at low temperature and is suitable to be used in following mixing steps. On the other hand, when the number-average molecular weight of PCL-PEG-PCL is too large and the ratio of the number-average molecular weight of PCL to that of PEG is not 3:2, it is unable to be applied to human bodies or having a physical property that only can be used at higher concentration. Some hydrogels even keep in gel phase no matter how the concentration or the temperature changes (not shown in figure) and thus have no effects.

Besides the hydrogel body, the hydrogel for treatment of cancers of the present invention also contains a plurality of anticancer drug molecules. The anticancer drug molecules can be radiopharmaceuticals or chemotherapeutic agents. According to users' needs, both radionuclides and chemotherapeutic agents are encapsulated in the hydrogel body.

The radiopharmaceuticals are radioactive isotopes, especially to those whose half-lives, difficulty in preparation, radiation type and strength are suitable for staying in the human body and applications of brachytherapy. The radioactive isotopes used in the present invention include Rhenium-188 (¹⁸⁸Re, 16.9 hours half life), Rhenium-186 (¹⁸⁶Re, 90 hours half life), Lutetium-177 (¹⁷⁷Lu, 6.7 days half life), Samarium-153 (¹⁵³Sm, 46.7 days half life), Iodine-131 (¹³¹I, 8 days half life), Indium-111(¹¹¹In, 67.9 hours half life), Yttrium-90 (⁹⁰Y, 64 hours half life), and Copper-64(⁶⁴Cu, 12.8 hours half life).

The chemotherapeutic agents include Camptothecin derivatives, Paclitaxel, Doxorubicin, etc. The chemotherapeutic agents have been nanosized. The derivative of Camptothecin is 7-ethyl-10-hydroxycamptothecin (Sn-38) with antitumor effect.

The hydrogel for treatment of cancers is formed by radiopharmaceuticals or chemotherapeutic agents coated with PCL-PEG-PC synthesized at a specific ratio and is characterized in thermal sensibility. As mentioned in above, the thermosensitive hydrogel at low concentration is gel at normal body temperature of 37° C. but liquid at lower temperature. For example, when the concentration of PCL-PEG-PCL of the present invention is 10%, it is liquid like aqueous solution when the environmental temperature is lower than 18° C. Utilizing temperature change, radiopharmaceuticals or chemotherapeutic agents covered with PCL-PEG-PCL is in liquid phase in catheters and this is convenient in operation of local injection against tumor tissues or hepatic artery embolization against liver cancers. After being infused into the treated area, the phase of PCL-PEG-PCL changes into gel-like solid and this allows drug retention in the treatment area. Thus the radiopharmaceuticals or chemotherapeutic agents are restricted in this area, without diffusing into other normal tissues. Thus the radiation dose or chemical dose absorbed by the normal tissue is lower and the side effects are reduced or absent.

Under the condition that the gel stays in the limited area, the radioactive isotopes used as radiopharmaceuticals will not stop emitting radiation rays to kill cancer cells until they decade into the level as low as the background radiation level gradually. As to nano-compounds used as chemotherapeutic agents, they can also be released slowly along with the degradation of PCL-PEG-PCL. Thus the exposure time to the anticancer drug molecules is prolonged and the cancer therapeutic efficacy is enhanced.

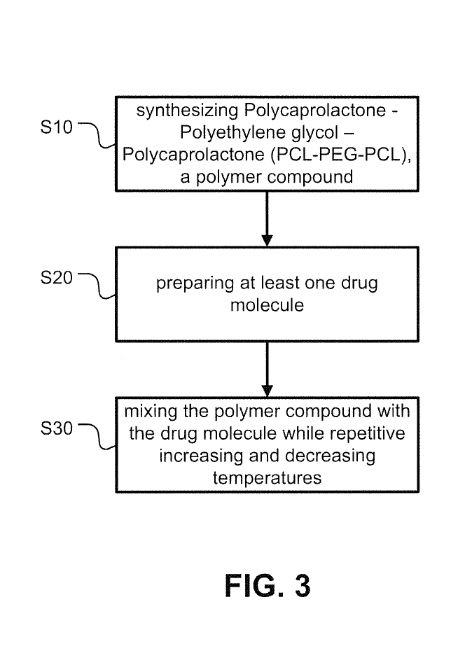

Refer to FIG. 3, a method for preparing thermosensitive hydrogel for treatment of cancers includes a plurality of steps.

Step S10: synthesizing a polymer compound-Polycaprolactone-Polyethylene glycol-Polycaprolactone, PCL-PEG-PCL;
Step S20: preparing drug molecules; and
Step S30: mixing polymer compound with the drug molecule;
repetitive temperature increase and decrease are used during the mixing.

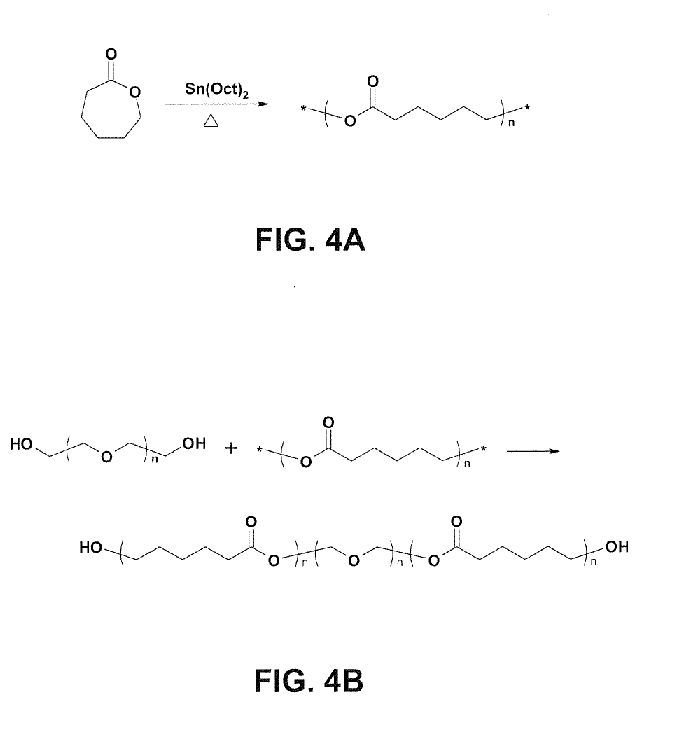

In the step S10, a synthesis method of PCL-PEG-PCL is revealed. Polyethylene glycol (PEG) is used as macroinitiator for ring-opening polymerization of ε-caprolactone so as to get PCL-PEG-PCL with the required molecular weight. Refer to FIG. 4A and FIG. 4B, the ring-opening polymerization of c-caprolactone is catalyzed by stannous octoate (Sn(Oct)₂) and also heated. Then the ring-opened caprolactone reacts with PEG to form the polymer compound PCL-PEG-PCL. In order to achieve the thermo-sensitivity, the ratio between PCL and PEG is limited. Thus the number-average molecular weight of the polymer compound is about 2500 and the ratio of the number-average molecular weight between PCL and PEG is 3:2.

The step S20 is to prepare drug molecules. This step is to make radiopharmaceuticals or chemotherapeutic agents have a coating of thermosensitive hydrogel. The drug molecules are radioisotope colloids or drugs in micelles (drugs delivered by using micellar nanoparticles). Both are coated with PCL-PEG-PCL independently or together with each other.

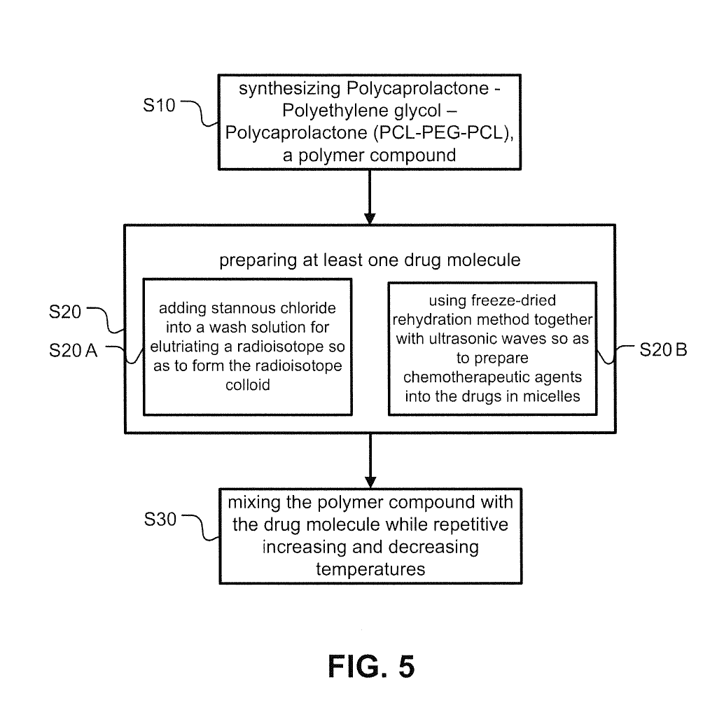

If the drug molecules being coated are radioisotope colloids, the method further includes a step of S20A. Refer to FIG. 5, stannous chloride is added into a washing solution for elutriating radioisotopes to form radioisotope colloids. The radioisotopes include those staying in the human body for a longer period and suitable for brachytherapy such as following nuclides ¹⁸⁸Re, ¹⁸⁶Re, ¹⁷⁷Lu, ¹⁵³Sm, ¹³¹I, ¹¹¹In, ⁹⁰Y, ⁶⁴Cu, etc.

If the drug molecules being coated are drugs in micelles, the method further includes a step of S20B. Refer to FIG. 5, the chemotherapeutic agents are prepared into drugs in micelles by freeze-dried rehydration method together with ultrasonic waves. The chemotherapeutic agent is selected from one of the followings: Camptothecin derivatives, Paclitaxel, and Doxorubicin. Besides, during synthesis of PCL-PEG-PCL, mPEG-PCL is also generated and this side chain structure could coat the micellar drugs properly.

After obtaining the polymer compound and the drug molecules, mix them. In the step S30, the temperature is increased over 70° C. and then decreased under 4° C. and this process is repeated so as to make them mix evenly. The drug molecules are distributed evenly in the polymer compound to get the thermosensitive hydrogel for treatment of cancers.

Take ¹⁸⁸Re used as drug molecules for cancer therapy as an example.

Step S10: synthesizing a polymer compound -Polycaprolactone-Polyethylene glycol-Polycaprolactone (PCL-PEG-PCL). Take 3.0 g polyethylene glycols, put it into a flask to be heated up to 120 degrees Celsius and residual water inside the polymer is removed by vacuum for four hours. Take 2.0 g c-caprolactone and 0.02 g stannous octoate and mix them together. Then raise temperature to 145° C. and stir constantly for 24 hours. Next dissolve the synthesized polymer in tetrahydrofuran(THF), ice-bathed in ethyl ether to have precipitate. After being filtered, the precipitate obtained is vacuum dried at room temperature.

Step S20: preparing a radioisotope colloid. Add 5-45 mg stannous chloride into perrhenate solution containing high purity of ¹⁸⁸Re (activity is about 370 MBq/ml) and heat the solution to 50° C. The heating time ranging from 5 to 120 minutes is adjusted according to the amount of stannous chloride added. Stop heating and cool the solution to room temperature. Then centrifuge, 3000 rpm for 10 minutes. Remove supernatant liquid and the residual obtained is radioisotope colloid.

Step S30: mixing the polymer compound with the radioisotope colloid and during the mixing process, the temperature is increased and decreased repetitively. The polymer compound and the radioisotope colloid obtained from the above steps are mixed and the temperature is increased up to 70° C. and then decreased to 4° C. at repeated cycles so as to make them mix evenly.

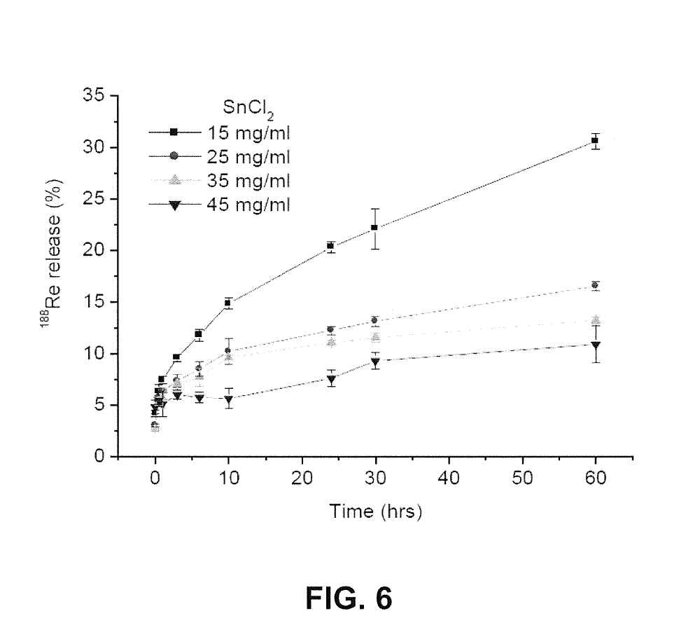

After in vitro tests and tests on animals with tumors, it is found that the release of ¹⁸⁸Re is quite slow. Refer to FIG. 6, the percent of ¹⁸⁸Re released from the area with tumor with time is quite low. Especially in the step S20A, the release ratio is the lowest when the concentration of stannous chloride added is 45 mg/ml. The half-life of ¹⁸⁸Re is 16.9 hours. As shown in FIG. 6, even after 3.5 half-lives, there is over 90% ¹⁸⁸Re stayed in the tumor area while the strength of ¹⁸⁸Re is reduced to 0.088 times of the original strength due to decay. Thus during the decay process of the radioisotope, the thermosensitive hydrogel for treatment of cancers and the anti-cancer drugs can stay on the treatment site for a longer period and the treatment effect is improved. The lifespan of test animals is prolonged and the growth of the tumor is inhibited.

In summary, the thermosensitive hydrogel of the present invention is produced after mixing and coating processes. Due to the thermosensitivity, the hydrogel provides continuous radiation or a slow releasing of the chemotherapeutic agents at the tumor site. The drug is highly concentrated in the tumor site and the reaction time is extended. After treatment, the hydrogel is degraded and metabolized gradually. Thus the hydrogel is an effective tool while being applied to cancer treatment.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A thermosensitive hydrogel for treatment of cancers comprising:
a hydrogel body which is Polycaprolactone-Polyethylene glycol-Polycaprolactone(PCL-PEG-PCL), a polymer compound; and
a plurality of anticancer drug molecules coated with the hydrogel body.

2. The thermosensitive hydrogel as claimed in claim 1, wherein number-average molecular weight of the polymer compound is about 2500 while a ratio of the number-average molecular weight between PCL and PEG is 3:2.

3. The thermosensitive hydrogel as claimed in claim 1, wherein the anticancer drug molecules are radiopharmaceuticals, chemotherapeutic agents or their combinations.

4. The thermosensitive hydrogel as claimed in claim 3, wherein the radiopharmaceutical is a radioisotope.

5. The thermosensitive hydrogel as claimed in claim 3, wherein the chemotherapeutic agents are selected from the group consisting Camptothecin derivatives, Paclitaxel, and Doxorubicin.

6. The thermosensitive hydrogel as claimed in claim 4, wherein the radioisotope is selected from the group consisting Rhenium-188 (¹⁸⁸Re), Rhenium-186 (¹⁸⁶Re), Lutetium-177 (¹⁷⁷Lu), Samarium-153 (¹⁵³Sm), Iodine-131 (¹³¹I), Indium-111(¹¹¹In), Yttrium-90(⁹⁰Y), and Copper-64(⁶⁴Cu).

7. A method for preparing a thermosensitive hydrogel for treatment of cancers comprising the steps of:
synthesizing Polycaprolactone-Polyethylene glycol-Polycaprolactone (PCL-PEG-PCL), a polymer compound;
preparing at least one drug molecule; and
mixing the polymer compound with the drug molecule while repetitive increasing and decreasing temperatures;
wherein the drug molecule is selected from the group consisting a radioisotope colloid, a drug in micelles and their combinations.

8. The method as claimed in claim 7, wherein the step of preparing at least one drug molecule further includes a step of:
adding stannous chloride into a wash solution for elutriating a radioisotope so as to form the radioisotope colloid;
wherein the radioisotope is selected from the group consisting Rhenium-188 (¹⁸⁸Re), Rhenium-186 (¹⁸⁶Re), Lutetium-177 (¹⁷⁷Lu), Samarium-153 (¹⁵³ Sm), Iodine-131 (¹³¹I), Indium-111(¹¹¹In), Yttrium-90(⁹⁰Y), and Copper-64(⁶⁴Cu).

9. The method as claimed in claim 7, wherein the step of preparing at least one drug molecule further includes a step of:
using freeze-dried rehydration method together with ultrasonic waves so as to prepare chemotherapeutic agents into the drugs in micelles;
wherein the chemotherapeutic agents are selected from the group consisting Camptothecin derivatives, Paclitaxel, and Doxorubicin.

10. The method as claimed in claim 7, wherein number-average molecular weight of the polymer compound is about 2500 while a ratio of the number-average molecular weight between PCL and PEG is 3:2.
